(19) United States
(12) Patent Application Publication
(10) Pub. No.: US 2026/0290517 A1
(43) Pub. Date: Sep. 24, 2026

(54) METHODS AND SYSTEMS FOR PREDICTING CHEMICAL REACTION PATHS

(71) Applicant: Good Chemistry Inc., Vancouver (CA)

(72) Inventors: Andrew Wildman, Tarrytown, NY (US); Takeshi Yamazaki

(21) Appl. No.: 19/476,537

(22) PCT Filed: Apr. 17, 2024
(86) PCT No.: PCT/IB2024/053746

§ 371 Date: Oct. 17, 2025

Related U.S. Application Data
(60) Provisional application No. 63/496,668, filed on Apr. 17, 2023.

(51) Int. Cl. G16C 20/10; G06N 20/20; G16C 20/70

(52) U.S. Cl. CPC G16C 20/10; G06N 20/20; G16C 20/70

(57) ABSTRACT

A computer-implemented method for determining a reaction path may include: (a) providing an indication of a reactant and at least one of a product or a driving coordinate; (b) providing a set of conformational coordinates, wherein said set of conformational coordinates is on a potential energy surface connecting said reactant and said at least one of said product or said driving coordinate; (c) using a trained model to evaluate an energy or a force at a conformational coordinate of said set of conformation coordinates; (d) determining a reliability metric at said conformational coordinate; and, optionally, (e) evaluating an ab initio energy or an ab initio force at said conformational coordinate.

### CROSS-REFERENCE

This application claims the benefit of U.S. Provisional Application Ser. No. 63/496,668, filed Apr. 17, 2023, which is incorporated herein by reference for all purposes.

### BACKGROUND

Chemical reactions are essential processes in creating molecules and materials. Therefore, prediction of chemical reactions has significant value in the discovery process for new molecules and materials in a wide variety of industries ranging from pharmaceutical, advanced material, and energy industries. Computational chemistry is an established tool for predicting a chemical reaction path.

There are various scenarios in which researchers try to predict the chemical reactions. As a first example, they may want to know if molecules (reactants) they have can cause chemical reactions and, if so, what kind of reaction path those molecules follow and/or what kind of product they will get as a result of a chemical reaction. As a second example, a researcher may know the molecules (reactants) that cause chemical reactions, and also know the product, but they may want to know properties of the potential energy surface defining the reaction such as the reaction path and/or activation barrier.

In either example, the above computational approaches may generate intermediate molecular geometries starting from reactants toward the geometry of potential products.

### SUMMARY

There are many approaches to determining intermediate molecular geometries and follow the trajectory of molecules in a chemical reaction. However, such calculations may be computationally expensive and/or inaccurate. Accuracy may be particularly useful around the transition state. Density functional theory (DFT) is an example method, but in some cases, DFT fails and a more accurate level of theory such as coupled cluster may be implemented. However, running high accuracy computations such as coupled cluster may be computationally expensive. As another example, classical force field models can describe bond forming and breaking at the level of a classical force field, such as ReaxFF, but their applicability may be limited by the force field parameters.

The present disclosure provides methods and systems for studying chemical reaction paths to overcome at least one of the above-identified drawbacks.

The present disclosure provides a computational chemistry platform that can predict a chemical reaction path accurately and efficiently by flexibly switching the energy and force solver between quantum chemistry or other chemical potential methods, such as DFT, CCSD (T), iFCI, QM/MM, DMET, ONIOM, and force field based potentials, and a machine learning (ML) model trained to the level of the quantum chemistry or chemical potential method, depending on the reliability of the inference that the ML model provides. If the platform indicates that the ML inference for a geometry along the reaction path is reliable, the platform continues using the ML model for that geometry. But when the platform indicates that the ML inference is less reliable, the platform may switch to a quantum chemistry method to solve the target geometry. In this way, the present disclosure increases both accuracy and efficiency.

In another example of increasing accuracy and efficiency, the results obtained from the quantum chemistry calculations may be stored in the platform's database as the training dataset to improve the ML model. In this way, the platform may reduce the number of times the quantum chemistry methods are used to solve the target geometry, and the platform may continuously improve the ML model to predict chemical reactions faster and more accurately.

In an aspect, the present disclosure provides a computer-implemented method for determining a reaction path. The method may comprise: (a) providing an indication of a reactant and at least one of a product or a driving coordinate; (b) providing a set of coordinates, wherein said set of coordinates is on an energy surface connecting said reactant and said at least one of said product or said driving coordinate; (c) evaluating an energy or a force at a coordinate of said set of coordinates using a trained model; (d) determining that a reliability metric at said coordinate is less than a threshold reliability value; (e) evaluating an energy or a force at said coordinate based at least in part on a quantum chemistry calculation corresponding to a training data set of said trained model; and (f) outputting a set of energies or forces at said set of coordinates on said energy surface based at least in part on said energy or force in (e) and said energy or said force in (c).

In some embodiments, energy surface is a potential energy surface. In some embodiments, said energy surface is a free energy surface. In some embodiments, said set of coordinates are a conformation coordinate. In some embodiments, said set of coordinates are cartesian coordinates. In some embodiments, said cartesian coordinates comprise a direction of movement. In some embodiments, (e) comprises evaluating an ab initio energy or an ab initio force at said coordinate.

In some embodiments, the method further comprises, until a completion criterion is met: (i) if said reliability metric is less than said threshold reliability value: evaluating said energy or said force at said coordinate in (c), selecting another coordinate on said energy surface, using said trained model to evaluate said energy or said force at said another coordinate, and determining a reliability metric for said energy or said force at said another coordinate; and (ii) if said reliability metric is greater than said threshold reliability value: selecting another coordinate on said energy surface, using said trained model to evaluate said energy at said another coordinate, and determining a reliability metric for said energy or said force at said another coordinate. In some embodiments, at (i), the method further comprises saving said energy or force for retraining and retraining said trained model based on said energy or said force in (i). In some embodiments, selecting said another coordinate on said energy surface comprises a method selected from the group consisting of ab initio molecular dynamics, nudged elastic band, growing string method, a variational reaction path optimization method, and intrinsic reaction coordinate.

In some embodiments, the method further comprises at (e) saving said energy or said force for retraining and retraining said trained model based on said energy or said force in (c). In some embodiments, the method further comprises using said trained model to evaluate a first energy or a first force at an initial coordinate. In some embodiments, the method further comprises determining a reliability metric for said first energy or said first force at said initial coordinate. In some embodiments, (f) further comprises outputting a transition state or a reaction path on said energy surface. In some embodiments, said reaction path is a minimum energy path.

In some embodiments, providing a set of coordinates in (b) comprises a method selected from the group consisting of ab initio molecular dynamics, nudged elastic band, growing string method, a variational reaction path optimization method, and intrinsic reaction coordinate. In some embodiments, said trained model comprises a machine learning algorithm. In some embodiments, said machine learning algorithm comprises a neural network. In some embodiments, said machine learning algorithm comprises an ensemble learning method. In some embodiments, (d) comprises combining results from one or more sub-models to create a metamodel, calculating an energy for each of said one or more sub-models, and calculating a standard deviation of energy for said one or more sub-models, wherein said standard deviation comprises a part of said reliability metric. In some embodiments, said metamodel comprises an ANI deep learning potential. In some embodiments, said one or more sub-models comprises one or more of PAINN deep learning potentials, DimeNet++ deep learning potentials, or PauliNet.

In some embodiments, (e) comprises evaluating an ab initio energy or an ab initio force at said coordinate, and wherein said ab initio energy or said ab initio force is calculated by a Hartree-Fock method, a coupled cluster method, full configuration interaction, incremental full configuration interaction, density functional theory, Moller-Plesset perturbation theory, mixed quantum mechanical and molecular mechanical methods, density matrix embedding theory, or ONIOM models.

In another aspect, the present disclosure provides a computer-implemented method for determining a reaction path. The method may comprise: (a) providing an indication of a reactant and at least one of a product or a driving coordinate; (b) providing a set of coordinates, wherein said set of coordinates is on an energy surface connecting said reactant and said at least one of said product or said driving coordinate; (c) evaluating an energy or a force at a coordinate of said set of coordinates using a trained model; (d) evaluating an energy or a force at said coordinate based at least in part on a quantum chemistry calculation corresponding to a training data set of said trained model; and (e) retraining said trained model based on said energy or said force in (d).

In some embodiments, said energy surface is a potential energy surface. In some embodiments, said energy surface is a free energy surface. In some embodiments, said set of coordinates are a conformation coordinate. In some embodiments, said set of coordinates are cartesian coordinates. In some embodiments, said cartesian coordinates comprise a direction of movement. In some embodiments, (d) comprises evaluating an ab initio energy or an ab initio force at said coordinate. In some embodiments, the method further comprises: outputting a set of energies or forces at said set of coordinates on said energy surface based at least in part on said energy or force in (d).

In some embodiments, said trained model comprises a machine learning algorithm. In some embodiments, said machine learning algorithm comprises a neural network. In some embodiments, said machine learning algorithm comprises an ensemble learning method. In some embodiments, (c) comprises: combining results from one or more sub-models to create a metamodel, calculating an energy for each of said one or more sub-models, and calculating a standard deviation of energy for said one or more sub-models, wherein said standard deviation comprises a reliability metric. In some embodiments, said metamodel comprises an ANI deep learning potential. In some embodiments, said one or more sub-models comprises one or more of PAINN deep learning potentials, DimeNet++ deep learning potentials, or PauliNet. In some embodiments, (d) comprises evaluating an ab initio energy or an ab initio force at said coordinate, and wherein said ab initio energy or said ab initio force is calculated by a Hartree-Fock method, a coupled cluster method, full configuration interaction, incremental full configuration interaction, density functional theory, Moller-Plesset perturbation theory, mixed quantum mechanical and molecular mechanical methods, density matrix embedding theory, or ONIOM models.

In some embodiments, the method further comprises: until a completion criterion is met: (i) if a reliability metric is less than said threshold reliability value: evaluating said energy or said force at said coordinate in (c), selecting another coordinate on said energy surface, using said trained model to evaluate said energy or said force at said another coordinate, and determining a reliability metric for said energy or said force at said another coordinate; and (ii) if a reliability metric is greater than said threshold reliability value: selecting another coordinate on said energy surface, using said trained model to evaluate said energy at said another coordinate, and determining a reliability metric for said energy or said force at said another coordinate. In some embodiments, the method further comprises at (i) saving said energy or force for retraining and retraining said trained model based on said energy or said force in (i).

In some embodiments, the method further comprises using said trained model to evaluate a first energy or a first force at an initial coordinate. In some embodiments, the method further comprises determining a reliability metric for said first energy or said first force at said initial coordinate. In some embodiments, said providing a set of coordinates in (b) comprises a method selected from the group consisting of: ab initio molecular dynamics, nudged elastic band, growing string method, a variational reaction path optimization method, and intrinsic reaction coordinate. In some embodiments, said selecting said another coordinate on said energy surface comprises a method selected from the group consisting of: ab initio molecular dynamics, nudged elastic band, growing string method, a variational reaction path optimization method, and intrinsic reaction coordinate.

In another aspect, the present disclosure provides a computer-implemented method for determining a reaction path. The method may comprise: (a) providing an indication of a reactant and at least one of a product or a driving coordinate; (b) providing a set of coordinates, wherein said set of coordinates is on an energy surface connecting said reactant and said at least one of said product or said driving coordinate; (c) providing a threshold reliability value for an energy or a force at a coordinate of said set of coordinates and, until a completion criterion is met: (i) if a reliability metric is less than said threshold reliability value: evaluating an energy or a force at said coordinate, selecting another coordinate on said energy surface, using a trained model to evaluate said energy or said force at said another coordinate, and determining said reliability metric for said energy or said force at said another coordinate; and (ii) if said reliability metric is greater than said threshold reliability value: selecting another coordinate on said potential energy surface, using said trained model to evaluate said energy at said another coordinate, and determining a reliability metric for said energy or said force at said another coordinate; and (d) outputting a set of energies or forces at said set of coordinates on said energy surface.

In some embodiments, said energy surface is a potential energy surface. In some embodiments, said energy surface is a free energy surface. In some embodiments, said set of coordinates are a conformation coordinate. In some embodiments, said set of coordinates are cartesian coordinates. In some embodiments, said cartesian coordinates comprise a direction of movement. In some embodiments, (c) comprises evaluating an ab initio energy or an ab initio force at said coordinate.

In some embodiments, the method further comprises retraining said trained model based on at least one energy or at least one force within set said set of coordinates. In some embodiments, the method further comprises said trained model to evaluate a first energy or a first force at an initial coordinate. In some embodiments, the method further comprises determining a reliability metric for said first energy or said first force at said initial coordinate. In some embodiments, (f) further comprises outputting a transition state or a reaction path on said energy surface. In some embodiments, said reaction path is a minimum energy path. In some embodiments, said providing a set of coordinates in (b) comprises a method selected from the group consisting of: ab initio molecular dynamics, nudged elastic band, growing string method, a variational reaction path optimization method, and intrinsic reaction coordinate. In some embodiments, said selecting said another coordinate on said energy surface comprises a method selected from the group consisting of: ab initio molecular dynamics, nudged elastic band, growing string method, a variational reaction path optimization method, and intrinsic reaction coordinate.

In some embodiments, said trained model comprises a machine learning algorithm. In some embodiments, said machine learning algorithm comprises a neural network. In some embodiments, said machine learning algorithm comprises an ensemble learning method. In some embodiments, (d) comprises: combining results from one or more sub-models to create a metamodel, calculating an energy for each of said one or more sub-models, and calculating a standard deviation of energy for said one or more sub-models, wherein said standard deviation comprises a part of said reliability metric. In some embodiments, said metamodel comprises an ANI deep learning potential. In some embodiments, said one or more sub-models comprises one or more of PAINN deep learning potentials, DimeNet++ deep learning potentials, or PauliNet. In some embodiments, (e) comprises evaluating an ab initio energy or an ab initio force at said coordinate, and wherein said ab initio energy or said ab initio force is calculated by a Hartree-Fock method, a coupled cluster method, full configuration interaction, incremental full configuration interaction, density functional theory, Moller-Plesset perturbation theory, mixed quantum mechanical and molecular mechanical methods, density matrix embedding theory, or ONIOM models.

In another aspect, the present disclosure provides a computer-implemented method for determining a reaction path. The method may comprise: (a) providing an indication of a reactant and at least one of a product or a driving coordinate; (b) providing a set of conformational coordinates, wherein said set of conformational coordinates is on a potential energy surface connecting said reactant and said at least one of said product or said driving coordinate; (c) using a trained model to evaluate an energy or a force at a conformational coordinate of said set of conformation coordinates; (d) determining that a reliability metric at said conformational coordinate is less than a threshold reliability value; (e) evaluating an ab initio energy or an ab initio force at said conformational coordinate; and (f) outputting a set of energies or forces at said set of conformational coordinates on said potential energy surface based at least in part on said ab initio energy or ab initio force and said energy or said force in (c).

In some embodiments, the method further comprises: until a completion criterion is met: (i) if said reliability metric is less than said threshold reliability value: evaluating said ab initio energy or said ab initio force at said conformational coordinate, selecting another conformation coordinate on said potential energy surface, using said trained model to evaluate said energy or said force at said another conformational coordinate, and determining a reliability metric for said energy or said force at said another conformational coordinate; and (ii) if said reliability metric is greater than said threshold reliability value: selecting another conformational coordinate on said potential energy surface, using said trained model to evaluate said energy at said another conformational coordinate, and determining a reliability metric for said energy or said force at said another conformational coordinate. In some embodiments, the method further comprises: at (e) saving said ab initio energy or said ab initio force for retraining and retraining said trained model based on said ab initio energy or said ab initio force. In some embodiments, the method further comprises: at (i) saving said ab initio energy or said ab initio force for retraining and retraining said trained model based on said ab initio energy or said ab initio force.

In some embodiments, the method further comprises: using said trained model to evaluate a first energy or a first force at an initial conformational coordinate. In some embodiments, the method further comprises: determining a reliability metric for said first energy or said first force at said initial conformational coordinate. In some embodiments, (f) further comprises outputting a transition state or a reaction path on said potential energy surface. In some embodiments, said reaction path is a minimum energy path. In some embodiments, said providing a set of conformational coordinates in (b) comprises a method selected from the group consisting of: ab initio molecular dynamics, nudged elastic band, growing string method, a variational reaction path optimization method, and intrinsic reaction coordinate. In some embodiments, said selecting said another conformational coordinate on said potential energy surface comprises a method selected from the group consisting of: ab initio molecular dynamics, nudged elastic band, growing string method, a variational reaction path optimization method, and intrinsic reaction coordinate.

In some embodiments, said trained model comprises a machine learning algorithm. In some embodiments, said machine learning algorithm comprises a neural network. In some embodiments, said machine learning algorithm comprises an ensemble learning method. In some embodiments, (d) comprises: combining results from one or more sub-models to create a metamodel, calculating an energy for each of said one or more sub-models, and calculating a standard deviation of energy for said one or more sub-models, wherein said standard deviation comprises a part of said reliability metric. In some embodiments, said metamodel comprises an ANI deep learning potential. In some embodiments, said one or more sub-models comprises one or more of PAINN deep learning potentials, DimeNet++ deep learning potentials, or PauliNet. In some embodiments, said ab initio energy or said ab initio force is calculated by a Hartree-Fock method, a coupled cluster method, full configuration interaction, incremental full configuration interaction, density functional theory, Moller-Plesset perturbation theory, mixed quantum mechanical and molecular mechanical methods, density matrix embedding theory, or ONIOM models.

In another aspect, the present application provides a computer-implemented method for determining a reaction path. The method may comprise: (a) providing an indication of a reactant and at least one of a product or a driving coordinate; (b) providing a set of conformational coordinates, wherein said set of conformational coordinates is on a potential energy surface connecting said reactant and said at least one of said product or said driving coordinate; (c) using a trained model to evaluate an energy or a force at a conformational coordinate of said set of conformational coordinates; (d) evaluating an ab initio energy or an ab initio force at said conformational coordinate; and (e) retraining said trained model based on said ab initio energy or said ab initio force.

In some embodiments, the method further comprises: outputting a set of energies or forces at said set of conformational coordinates on said potential energy surface based at least in part on said ab initio energy or ab initio force. In some embodiments, said trained model comprises a machine learning algorithm. In some embodiments, said machine learning algorithm comprises a neural network. In some embodiments, said machine learning algorithm comprises an ensemble learning method. In some embodiments, (c) comprises: combining results from one or more sub-models to create a metamodel, calculating an energy for each of said one or more sub-models, and calculating a standard deviation of energy for said one or more sub-models, wherein said standard deviation comprises said reliability metric. In some embodiments, said metamodel comprises an ANI deep learning potential. In some embodiments, said one or more sub-models comprises one or more of PAINN deep learning potentials, DimeNet++ deep learning potentials, or PauliNet.

In another aspect, the present disclosure provides, a computer-implemented method for determining a reaction path. The method may comprise: (a) providing an indication of a reactant and at least one of a product or a driving coordinate; (b) providing a set of conformational coordinates, wherein said set of conformational coordinates is on a potential energy surface connecting said reactant and said at least one of said product or said driving coordinate; (c) providing a threshold reliability value for an energy or a force at a conformational coordinate of said set of conformational coordinates and, until a completion criterion is met: (i) if a reliability metric is less than said threshold reliability value: evaluating an ab initio energy or an ab initio force at said conformational coordinate, selecting another conformation coordinate on said potential energy surface, using a trained model to evaluate said energy or said force at said another conformational coordinate, and determining said reliability metric for said energy or said force at said another conformational coordinate; and (ii) if said reliability metric is greater than said threshold reliability value: selecting another conformational coordinate on said potential energy surface, using said trained model to evaluate said energy at said another conformational coordinate, and determining a reliability metric for said energy or said force at said another conformational coordinate; and (d) outputting a set of energies or forces at said set of conformational coordinates on said potential energy surface.

In some embodiments, the method further comprises retraining said trained model based on at least one ab initio energy or at least one ab initio force within set said set of conformational coordinates.

Another aspect of the present disclosure provides a system comprising one or more computer processors and computer memory coupled thereto. The computer memory comprises machine executable code that, upon execution by the one or more computer processors, implements any of the methods above or elsewhere herein.

In some embodiments, the system further comprises a quantum computer, wherein the quantum computer is configured to perform an operation of any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is a flowchart of an example of a method for determining a reaction path, in accordance with some embodiments.

FIG. 2 is a flowchart of another example of a method for determining a reaction path when the reliability is less than a threshold value, in accordance with some embodiments.

FIG. 3 is a flowchart of an example of a method for determining a reaction path and using data from a quantum chemistry calculation to update a trained model, in accordance with some embodiments.

FIG. 4 is a flowchart an example of a method for determining a reaction path showing branching pathways based on a reliability metric, in accordance with some embodiments.

FIG. 5, there is shown an example schematic diagram of a system for determining a reaction path, in accordance with some embodiments.

FIG. 6 shows a computer system that is programmed or otherwise configured to implement methods provided herein.

FIG. 7 is a diagram of a chemical reaction between s-cis-butadiene and ethene to form cyclohexene.

FIG. 8 is a plot of computational data showing: reaction path energy profiles for DFT (solid), ANI/ANI1x (dashed), and ANI/Transition1x (dotted); GSM node energies (open circles); and intermediate values estimated with a cubic spline interpolation (regression lines).

FIG. 9A is computational data showing molecular structures for the reactants (left), transition state (center), and products (right) predicted by GSM using the ANI/ANI1x model.

FIG. 9B is computational data showing molecular structures for the reactants (left), transition state (center), and products (right) predicted by GSM using the ANI/Transition1x model.

FIG. 9C is computational data showing molecular structures for the reactants (left), transition state (center), and products (right) predicted by GSM using the DFT model.

FIG. 10 shows overlaid transition state structures predicted by GSM using DFT, (filled, 1030) ANI/ANI1x, (open, 1010) and ANI/Transition1x. (textured, 1020), where all structures have been translated to have the same overall center of mass.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out.

The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be used, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

The present disclosure provides methods and systems for predicting reaction paths using a general machine learned (ML) potential that can detect when it is unreliable and switch to a reliable, equivalent ab initio calculation instead. An ML potential may be the potential generated from an ML model that takes in at least an indication of molecular conformation and returns at least the energy of that conformation. The energy and force from the ab initio calculation may be directly used in place of the ML potential, and the data may be saved for later retraining of the ML potential. When combined with overall reaction path prediction algorithms, this technique may form a robust and rapid method for calculating reaction paths and transition states that continuously improves the underlying ML model.

The present disclosure provides a computational chemistry platform that can predict a chemical reaction path accurately and efficiently by flexibly switching the energy and force solver between quantum chemistry or other chemical potential methods, such as DFT, CCSD (T), iFCI, QM/MM, DMET, ONIOM, and force field based potentials, and a machine learning (ML) model trained to the level of the quantum chemistry or chemical potential method depending on the reliability of the inference that the ML model provides. If the platform indicates that the ML inference for a geometry along the reaction path is reliable, the platform continues using the ML model for that geometry. But when the platform indicates that the ML inference is less reliable, the platform may switch to the quantum chemistry methods to solve the target geometry. In this way, the present disclosure increases both accuracy and efficiency.

In another example of increasing accuracy and efficiency, the results obtained from the quantum chemistry calculations may be stored in the platform's database as the training dataset to improve the ML model. In this way, the platform may reduce the number of times the quantum chemistry methods are used to solve the target geometry, and the platform may continuously improve the ML model to predict chemical reactions faster and more accurately.

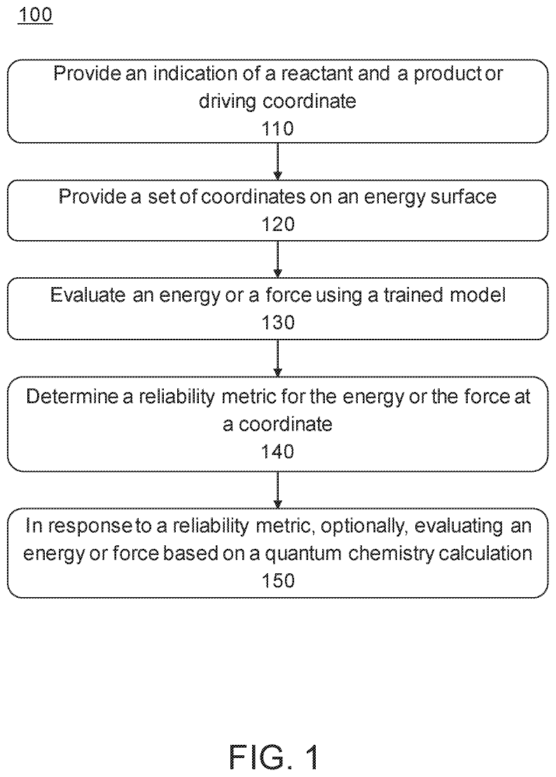

FIG. 1 is a flowchart of an example of a method 100 for determining a reaction path. At an operation 110, the method may comprise providing an indication of a reactant and a product or a driving coordinate. At an operation 120, the method may comprise providing a set of coordinates on an energy surface. At an operation 130, the method may comprise evaluating an energy or a force using a trained model. At an operation 140, the method may comprise determining a reliability metric for the energy or the force at the coordinate determined with the trained model. At an operation 150, in response to the reliability metric the method may comprise, optionally, evaluating an energy or a force based on a quantum chemistry calculation.

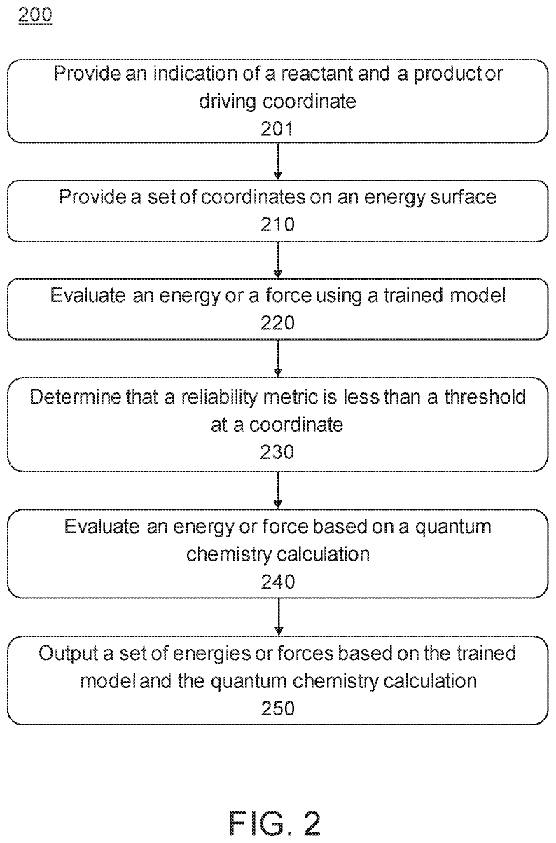

FIG. 2 is a flowchart of an example of a method 200 for determining a reaction path when the reliability is less than a threshold value. Method 200 may comprise an embodiment variation or example of method 100. At an operation 201, method 200 may comprise providing an indication of a reactant and at least one of a product or a driving coordinate. Operation 201 may comprise an embodiment, variation, or example of operation 110 of the method 100. At an operation 210, method 200 may comprise providing a set of coordinates. The set of coordinates may be on an energy surface connecting the reactant and at least one of the product or the driving coordinate. Operation 210 may comprise an embodiment, variation, or example of operation 120 of the method 100. At an operation 220, method 200 may comprise evaluating an energy or a force at a coordinate of the set of coordinates using a trained model. Operation 220 may comprise an embodiment, variation, or example of operation 130 of the method 100. At an operation 230, method 200 may comprise determining that a reliability metric at the coordinate is less than a threshold reliability value. Operation 230 may comprise an embodiment, variation, or example of operation 140 of the method 100 when the reliability metric is less than a threshold. At an operation 240, method 200 may comprise evaluating an energy or a force at the coordinate based at least in part on a quantum chemistry calculation corresponding to a training data set of the trained model. Operation 240 may comprise an embodiment, variation, or example of operation 150 of the method 100 when the energy or the force is calculated using the quantum chemistry calculation. At an operation 250, method 200 may comprise outputting a set of energies or forces at the set of coordinates on the energy surface based at least in part on the energy or force in 240 and the energy or the force in operation 220.

In an example of the method 200, the present disclosure provides a computer-implemented method for determining a reaction path. The method may comprise: (a) providing an indication of a reactant and at least one of a product or a driving coordinate; (b) providing a set of conformational coordinates, wherein the set of conformational coordinates is on a potential energy surface connecting the reactant and the at least one of the product or the driving coordinate; (c) using a trained model to evaluate an energy or a force at a conformational coordinate of the set of conformation coordinates; (d) determining that a reliability metric at the conformational coordinate is less than a threshold reliability value; I evaluating an ab initio energy or an ab initio force at the conformational coordinate; and (f) outputting a set of energies or forces at the set of conformational coordinates on the potential energy surface based at least in part on the ab initio energy or ab initio force and the energy or the force in (c).

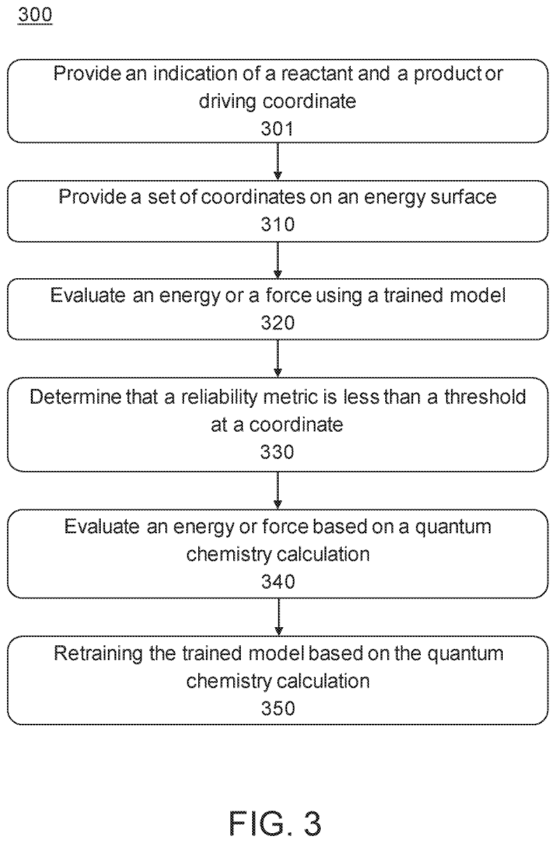

FIG. 3 is a flowchart of an example of a method 300 for determining a reaction path and using data from a quantum chemistry calculation to update a trained model. Method 300 may comprise an embodiment variation or example of method 100. At an operation 301, method 300 may comprise providing an indication of a reactant and at least one of a product or a driving coordinate. Operation 301 may comprise an embodiment, variation, or example of operation 110 of the method 100. At an operation 310, method 300 may comprise providing a set of coordinates. The set of coordinates may be on an energy surface connecting the reactant and at least one of the product or the driving coordinate. Operation 310 may comprise an embodiment, variation, or example of operation 320 of the method 100. At an operation 320, method 300 may comprise evaluating an energy or a force at a coordinate of the set of coordinates using a trained model. Operation 320 may comprise an embodiment, variation, or example of operation 130 of the method 100. At an operation 330, method 300 may comprise determining that a reliability metric at the coordinate is less than a threshold reliability value. Operation 330 may comprise an embodiment, variation, or example of operation 140 of the method 100 when the reliability metric is less than a threshold. At an operation 340, method 300 may comprise evaluating an energy or a force at the coordinate based at least in part on a quantum chemistry calculation corresponding to a training data set of the trained model. Operation 340 may comprise an embodiment, variation, or example of operation 150 of the method 100 when the energy or the force is calculated using the quantum chemistry calculation. At an operation 350, method 300 may comprise retraining the trained model based on the energy or the force in 340.

In an example of the method 300, the present application provides a computer-implemented method for determining a reaction path. The method may comprise: (a) providing an indication of a reactant and at least one of a product or a driving coordinate; (b) providing a set of conformational coordinates, wherein the set of conformational coordinates is on a potential energy surface connecting the reactant and the at least one of the product or the driving coordinate; (c) using a trained model to evaluate an energy or a force at a conformational coordinate of the set of conformational coordinates; (d) evaluating an ab initio energy or an ab initio force at the conformational coordinate; and (e) retraining the trained model based on the ab initio energy or the ab initio force.

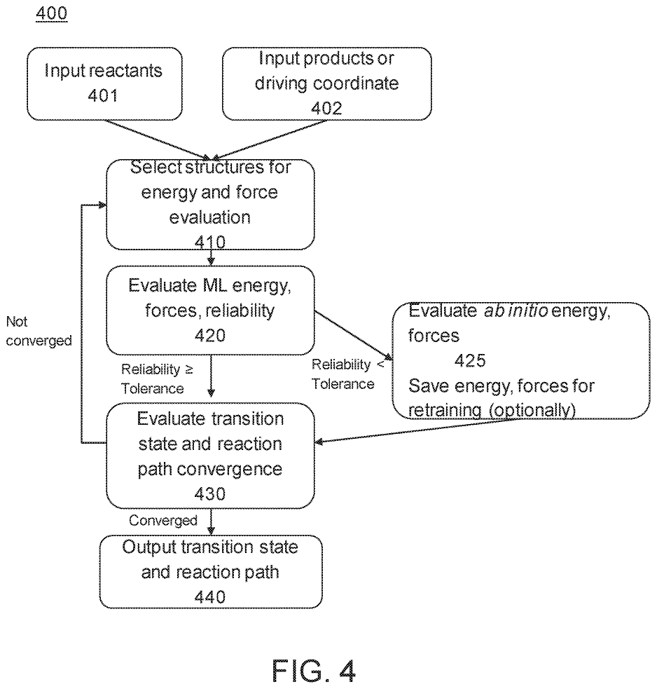

FIG. 4 is a flowchart an example of a method 400 for determining a reaction path showing branching pathways based on a reliability metric. Method 400 may comprise an embodiment variation or example of method 100.

At an operation 401, method 400 may comprise providing an indication of a reactant. At an operation 402, method 400 may comprise providing at least one of a product or a driving coordinate. The indication at operation 401, operation 402, or both may be inputs to the computer implemented methods described herein, for example, operation 110 of a method 100. An operation 401, operation 402, or both may comprise providing an indication of a reactant and at least one of a product or a driving coordinate. At an operation 410, method 400 may comprise selecting structures for energy evaluation, force evaluation, or both. An operation 410 may comprise providing a set of coordinates, wherein the set of coordinates is on an energy surface connecting the reactant and the at least one of the product or the driving coordinate. At an operation 420, method 400 may comprise evaluating an energy or a force at a coordinate of the set of coordinates using a trained model. Operation 420 may comprise an embodiment, variation, or example of operation 130 of the method 100. At an operation 420, method 400 may further comprise determining a reliability metric at the coordinate. Operation 420 may comprise an embodiment, variation, or example of operation 140 of the method 100. After an operation 420, method 400 may branch based on a reliability. In some cases, method 400 may comprise providing a threshold reliability value for an energy or a force at a coordinate of the set of coordinates. If a reliability metric is less than the threshold reliability value, the method 400 may proceed to operation 425. If a reliability metric is greater than the threshold reliability value, the method 400 may proceed to operation 435. At an operation 425, method 400 may comprise evaluating an energy or a force at the coordinate based at least in part on a quantum chemistry calculation corresponding to a training data set of the trained model. Operation 340 may comprise an embodiment, variation, or example of operation 150 of the method 100 when the energy or the force is calculated using the quantum chemistry calculation. selecting another coordinate on the energy surface, using a trained model to evaluate the energy or the force at another coordinate, and determining the reliability metric for the energy or the force at another coordinate. At an operation 430, method 400 may comprise, if the reliability metric is greater than the threshold reliability value, selecting another coordinate on the potential energy surface, using the trained model to evaluate the energy at another coordinate, and determining a reliability metric for the energy or the force at another coordinate. In some cases, method 400 may comprise repeating 410, 420, 425, and 430 until a stopping criterion is met. At an operation 440, method 400 may comprise outputting a set of energies or forces at the set of coordinates on the energy surface based at least in part on the energy or force in 430.

In an example of method 400, the present disclosure provides, a computer-implemented method for determining a reaction path. The method may comprise: (a) providing an indication of a reactant and at least one of a product or a driving coordinate; (b) providing a set of conformational coordinates, wherein the set of conformational coordinates is on a potential energy surface connecting the reactant and the at least one of the product or the driving coordinate; (c) providing a threshold reliability value for an energy or a force at a conformational coordinate of the set of conformational coordinates and, until a completion criterion is met: (i) if a reliability metric is less than the threshold reliability value: evaluating an ab initio energy or an ab initio force at the conformational coordinate, selecting another conformation coordinate on the potential energy surface, using a trained model to evaluate the energy or the force at the another conformational coordinate, and determining the reliability metric for the energy or the force at the another conformational coordinate; and (ii) if the reliability metric is greater than the threshold reliability value: selecting another conformational coordinate on the potential energy surface, using the trained model to evaluate the energy at the another conformational coordinate, and determining a reliability metric for the energy or the force at the another conformational coordinate; and (d) outputting a set of energies or forces at the set of conformational coordinates on the potential energy surface.

### Reaction Path Sampling and Initialization

Reaction path sampling, sometimes called transition path sampling, may comprise a form of simulation in which trajectories of physical or chemical transitions of a system from one stable state to other are calculated. Examples of target systems for reaction path sampling include protein folding, chemical reactions, crystal nucleation, etc. Of interest in a reaction path sampling calculation is a transition state. In a simple example, a state A and a state B may be stable states of a system. State A and State B may be connected by a trajectory along a reaction path. The reaction may be characterized by a local energy maximum between the stable states. The local energy maximum may be the transition state. The transition state may be characterized by an activation energy barrier. In some cases, the reaction path may be characterized by an ensemble of transition paths. The transition state geometry and the activation energy barrier may be of interest in understanding chemical reactions (e.g., what chemical reactions happen) and rate constants (e.g., how fast they happen). These calculations may help generate understanding of important phenomena like docking a ligand at a protein, per- and polyfluoroalkyl substances (PFAS) reactions, etc.

However, these types of calculation may be difficult to perform. The transition state itself is a non-equilibrium state which may be difficult to model quantum chemical. Further, from a molecular dynamics perspective, the size of the ensemble of paths used to reach chemical accuracy may become quite large. As such, these may be promising systems for advanced quantum chemical methods.

Providing an indication of a reactant and a product or a driving coordinate: At an operation 110, the method may comprise providing an indication of a reactant and a product or a driving coordinate. Operation 201 may comprise an embodiment, variation, or example of operation 110 of the method 100. Operation 301 may comprise an embodiment, variation, or example of operation 110 of the method 100.

At an operation 401, method 400 may comprise providing an indication of a reactant. At an operation 402, method 400 may comprise providing at least one of a product or a driving coordinate. The indication at operation 401, operation 402, or both may be inputs to the computer implemented methods described herein, for example, operation 110 of a method 100. An operation 401, operation 402, or both may comprise providing an indication of a reactant and at least one of a product or a driving coordinate. The indication at operation 401, operation 402, or both may be provided by a user.

In some cases, the method may comprise providing an indication of a reactant. In some cases, the method may comprise providing an indication of a product or a driving coordinate. The indication of the reactant, the product, or both may comprise an indication of a chemical structure. In some cases, the structure is provided in a coordinate system. In some cases, the set of coordinates are a conformation coordinate. In some cases, the set of coordinates are cartesian coordinates. In some embodiments, the cartesian coordinates comprise a direction of movement. In some case, the indication of the reactant, the product, the driving coordinate or any combination of thereof comprise a simplified molecular-input line-entry system (SMILES) string. In some case, the indication of the reactant, the product, the driving coordinate or any combination of thereof comprise a 2D drawing. In some case, the indication of the reactant, the product, the driving coordinate or any combination of thereof comprise 3D model.

In some cases, the product may not be known at the start of a computation. In the case of an unknown product, a driving coordinate may be provided. A driving coordinate may comprise a bond bending coordinate, a bond breaking coordinate, a tortional coordinate, a stretching coordinate, a bending coordinate, a geometric distortion of the reactant of any kind, etc. In some cases, the driving coordinate is a direction of motion on a geometric coordinate system. In some cases, the driving coordinate is a direction of motion on energy coordinate system. In the case, where the product is unknown, the reaction path sampling calculation may be used to find the product.

The indication may be provided by a user. In some cases, the user provides the indication via an interface remote to a computing platform.

Providing a set of coordinates on an energy surface: At an operation 120, the method 100 may comprise providing a set of coordinates on an energy surface. Operation 210 may comprise an embodiment, variation, or example of operation 120 of the method 100. Operation 310 may comprise an embodiment, variation, or example of operation 320 of the method 100. At an operation 410, method 400 may comprise selecting structures for energy evaluation, force evaluation, or both. An operation 410 may comprise providing a set of coordinates, wherein the set of coordinates is on an energy surface connecting the reactant and the at least one of the product or the driving coordinate.

With the reactant and the product or the driving coordinate provided, the space within which the reaction path is to be determined may be provided. In some cases, the set of coordinates comprises a set of conformational coordinates. In some cases, the set of conformational coordinates is on an energy surface connecting the reactant and the at least one of the product or the driving coordinate. In some cases, energy surface is a potential energy surface. In some cases, the energy surface is a free energy surface.

In some cases, the method 100, 200, 300, or 400 may further comprise an operation of selecting a conformational coordinate within the set of conformational coordinates. In some cases, the method 100, 200, 300, or 400 may further comprise generating a first or initial coordinate. In some cases, the method 100, 200, 300, or 400 may further comprise using a trained model described herein below to evaluate a first energy or a first force at an initial coordinate. In some cases, the method 100, 200, 300, or 400 may further comprise determining a reliability metric for the first energy or the first force at the initial coordinate.

In some cases, providing a set of coordinates at 120 comprises a method selected from the group consisting of: ab initio molecular dynamics, nudged elastic band, growing string method, a variational reaction path optimization method, and intrinsic reaction coordinate. Any of the methods may be used to generate a set of coordinates and to move to a next coordinate within the set of coordinates. For example, there are various approaches to generate an intermediate or next molecular geometry and to follow the trajectory of molecules in the chemical reaction; such approaches include Ab Initio Molecular Dynamics (AIMD), NEB (Nudged Elastic Band), and GSM (Growing String Method), a variational reaction path optimization method, IRC (Intrinsic Reaction Coordinate) etc.

Reaction path prediction methods may seek to determine the minimum energy path (MEP) between a given point on the potential energy surface and some goal, e.g., a product, a next conformational state, a next molecular coordinate, etc. In double ended methods, the goal is another point on the potential energy surface. In single ended methods, this goal is the first saddle point approximately in a given direction on the potential energy surface.

There are many methods that can be used to compute the MEP. Some rely on sampling the surface via metadynamics, (e.g., Born Oppenheimer molecular dynamics) and some explore the surface more directly with string-based methods (e.g., nudged elastic band (NEB) or growing string method (GSM)). Others form a reaction path from a transition state (e.g., intrinsic reaction coordinate). Each of these example methods may be integrated into the general outline shown in FIGS. 1-4.

### Trained Model

At an operation 130, the method may comprise evaluating an energy or a force using a trained model. Operation 220 may comprise an embodiment, variation, or example of operation 130 of the method 100. Operation 320 may comprise an embodiment, variation, or example of operation 130 of the method 100. Operation 420 may comprise an embodiment, variation, or example of operation 130 of the method 100.

Because of the computational expense of certain computational methods, machine learning models have been proposed as alternatively for computationally expensive calculations. Machine learning (ML) methods may be used in various fields such as quantum chemistry and materials simulation. For example, ML methods may be used to deliver predictive models of interatomic potential energy surfaces, molecular forces, electron densities, density functionals, and molecular response properties such as polarizabilities and infrared spectra. Large data sets of molecular properties calculated from quantum chemistry or measured from experiment can both be used to construct predictive models to explore the vast chemical compound spaces, to find new sustainable catalyst materials, and to design new synthetic pathways. In some cases, machine learning can be used in constructing approximate quantum chemical methods, such as predicting MP2 and coupled cluster energies from Hartree-Fock orbitals. In some cases, neural networks may be used as a basis representation of the wavefunction. In general, ML models may learn from quantum chemistry datasets to describe molecular properties as scalar, vector, or tensor fields. In an example, quantum chemistry data of different electronic properties, such as energies or dipole moments, may be used to construct individual ML models for the respective properties. ML may allow for the efficient exploration of chemical space with respect to these properties.

Examples of Machine Learning Methodologies—In some cases, at an operation 130, the method may comprise evaluating an energy or a force using a trained model. In some cases, the trained model comprises a machine learning algorithm. In some cases, ML may generally involve identifying and recognizing patterns in existing data in order to facilitate making predictions for subsequent data. ML may include a ML model (which may include, for example, a ML algorithm). Machine learning, whether analytical or statistical in nature, may provide deductive or abductive inference based on real or simulated data. The ML model may be a trained model.

ML techniques may comprise one or more supervised, semi-supervised, self-supervised, or unsupervised ML techniques. For example, an ML model may be a trained model that is trained through supervised learning (e.g., various parameters are determined as weights or scaling factors). ML may comprise one or more of regression analysis, regularization, classification, dimensionality reduction, ensemble learning, meta learning, association rule learning, cluster analysis, anomaly detection, deep learning, or ultra-deep learning. ML may comprise, but is not limited to: k-means, k-means clustering, k-nearest neighbors, learning vector quantization, linear regression, non-linear regression, least squares regression, partial least squares regression, logistic regression, stepwise regression, multivariate adaptive regression splines, ridge regression, principal component regression, least absolute shrinkage and selection operation (LASSO), least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short-term memory, deep belief networks, deep Boltzmann machines, deep convolutional neural networks, deep recurrent neural networks, or generative adversarial networks.

Training Data—The trained model may be trained on a data set of quantum chemical information. For example, a dataset may comprise the Transition 1× data set, see https://arxiv.org/abs/2207.12858, which is incorporated herein by reference for all purposes. In some cases, a base data set may be supplemented by data from a user base. For example, prior use quantum chemical calculations may be used to generate a proprietary data set or to supplement an existing one. A training data set may comprise a coordinate and an energy for a set of reactants and products. In some cases, a training data set may also comprise forces. A data set may be more accurate the larger the number of samples which are similar to the data point to be calculated. In some cases, a data set with more data with structures similar to the transition state may be more accurate.

Training—Training the ML model may include, in some cases, selecting one or more untrained data models to train using a training data set. The selected untrained data models may include any type of untrained ML models for supervised, semi-supervised, self-supervised, or unsupervised machine learning. The selected untrained data models may be specified based upon input (e.g., user input) specifying relevant parameters to use as predicted variables or other variables to use as potential explanatory variables. For example, the selected untrained data models may be specified to generate an output (e.g., a prediction) based upon the input. Conditions for training the ML model from the selected untrained data models may likewise be selected, such as limits on the ML model complexity or limits on the ML model refinement past a certain point. The ML model may be trained (e.g., via a computer system such as a server) using the training data set. In some cases, a first subset of the training data set may be selected to train the ML model. The selected untrained data models may then be trained on the first subset of training data set using appropriate ML techniques, based upon the type of ML model selected and any conditions specified for training the ML model. In some cases, due to the processing power requirements of training the ML model, the selected untrained data models may be trained using additional computing resources (e.g., cloud computing resources). Such training may continue, in some cases, until at least one aspect of the ML model is validated and meets selection criteria to be used as a predictive model.

In some cases, one or more aspects of the ML model may be validated using a second subset of the training data set (e.g., distinct from the first subset of the training data set) to determine accuracy and robustness of the ML model. Such validation may include applying the ML model to the second subset of the training data set to make predictions derived from the second subset of the training data. The ML model may then be evaluated to determine whether performance is sufficient based upon the derived predictions. The sufficiency criteria applied to the ML model may vary depending upon the size of the training data set available for training, the performance of previous iterations of trained models, or user-specified performance requirements. If the ML model does not achieve sufficient performance, additional training may be performed. Additional training may include refinement of the ML model or retraining on a different first subset of the training dataset, after which the new ML model may again be validated and assessed. When the ML model has achieved sufficient performance, in some cases, the ML may be stored for present or future use. The ML model may be stored as sets of parameter values or weights for analysis of further input (e.g., further relevant parameters to use as further predicted variables, further explanatory variables, further user interaction data, etc.), which may also include analysis logic or indications of model validity in some instances. In some cases, a plurality of ML models may be stored for generating predictions under different sets of input data conditions. In some embodiments, the ML model may be stored in a database (e.g., associated with a server).

Neural Networks—In some embodiments, the machine learning algorithm comprises a neural network. One class of machine learning algorithms, artificial neural networks (ANNs), may comprise a portion of the trained model herein. For example, feedforward neural networks (such as convolutional neural networks or CNNs) and recurrent neural networks (RNNs) may be used. In some cases, multiple layers of neural networks may be employed, creating a deep neural network. Using a deep neural network may increase the predictive power of a neural network algorithm. In some cases, a machine learning algorithm using a neural network may further include Adam optimization (e.g., adaptive learning rate), regularization, etc. The number of layers, the number of nodes within the layer, a stride length in a convolutional neural network, a padding, a filter, etc. may be adjustable parameters in a neural network.

Ensemble Learning—In some embodiments, the machine learning algorithm comprises an ensemble learning method. Ensemble learning may be a machine learning technique that enhances accuracy and resilience in forecasting by merging predictions from multiple models.

Ensemble learning may create a large—scale metamodel by statistically combining the results from several smaller, simpler models. These smaller models may not need to have the same architecture, but they may predict the same or similar features to the larger model. In some cases, the output of the metamodel and each of the sub-models is the energy of the molecular system. The energy from the metamodel is the mean of the energies from the sub-models, which may be represented as

$${E}_{Meta} = {{\sum }_{i = 1}}^{L} {E}_{i}$$

where L is the total number of sub-models in the ensemble.

In some cases, the ensemble learning method comprises an ANI architecture. A description of the ANI architecture is provided at: https://arxiv.org/ftp/arxiv/papers/1801/1801.09319.pdf, which is incorporated herein by reference for all purposes. If the reliability criterion is larger than a tolerance, then that structure may be used for retraining of the model.

The ANI model may take in the atomic numbers and coordinates in cartesian space, and it may predict an energy for the whole molecule. An example using ANI-1 is shown at, for example, https://arxiv.org/ftp/arxiv/papers/1610/1610.08935.pdf, which is incorporated herein by reference for all purposes.

The ANI model belongs to the family of general machine learned chemical potential methods. The ANI model is not specific to any particular atom or system type. However, in some cases, specific trained instances of it, such as ANI-1x and ANI-2x may have a constrained set of allowed atoms. The ML model, such as for example the ANI model, may directly predict the energy of the molecular system. Additional details of the ANI model be found at, for may example, https://arxiv.org/ftp/arxiv/papers/1610/1610.08935.pdf, which is incorporated herein by reference for all purposes. Since the input parameters for the ANI model comprise atomic coordinates, one may find the force on the molecule through automatic differentiation.

In some cases, the ensemble learning method comprises combining results from one or more sub-models to create a metamodel. In some cases, the ensemble learning method comprises a MACE or an espaloma architecture. In some cases, a sub-model within the meta-model comprises MACE or espaloma. MACE may comprise an equivariant message passing neural network, see https://arxiv.org/pdf/2206.07697.pdf, which is incorporated by reference herein in its entirety. Espaloma may comprise machine-learned molecular mechanics force field, see https://arxiv.org/abs/2307.07085, which is incorporated by reference herein in its entirety. In some embodiments, a sub-model within the metamodel comprises an ANI deep learning potential. In some embodiments, the one or more sub-models comprises one or more of polarizable atom interaction neural network (PaiNN), DimeNet++ deep learning potentials, or PauliNet. PaiNN (a deep potential) learning is described at, for example, https://arxiv.org/abs/2102.03150, which is incorporated herein by reference in its entirety. DimeNet++ is described at, for example, https://arxiv.org/abs/2011.14115, which is incorporated herein by reference in its entirety. PauliNet is described at, for example, https://arxiv.org/abs/1909.08423, which is incorporated herein by reference in its entirety. In some case, the one or more sub-models comprises SchNET, OCP, etc. SchNET is described at, for example, https://arxiv.org/abs/1712.06113, which is incorporated herein by reference in its entirety. Open catalyst project (OCP) also produces an AI model which may be used as a sub-model herein, see, for example, https://opencatalystproject.org/, which is incorporated herein by reference in its entirety.

### Automated Reliability

At an operation 140, the method may comprise determining a reliability metric for the energy or the force at the coordinate determined with the trained model. In some cases, the method may comprise determining a reliability metric. A trained model and reaction path prediction may be combined to simultaneously provide highly reliable reaction path prediction and transition state determination. In some cases, the method may also continuously improve the ML model used to provide the potential along this path. For example, a reaction path prediction algorithm may be used to generate structures for investigation, which produces a set of molecular coordinates. Next, an ensemble general ML potential may be used to evaluate the energy, forces, and reliability at this set of molecular coordinates.

From there, two things may occur. If the ML potential is reliable enough, (e.g., the standard deviation is lower than an acceptance threshold), these energies and forces may be directly passed back to the reaction path prediction algorithm. If the ML potential is not reliable enough, a quantum chemistry calculation may be launched to calculate energies and forces ab initio. The type of quantum chemistry calculation launched may match that used to generate the original training dataset of the ML potential. For example, if the original training set was calculated using coupled cluster singles and doubles with the cc-pVTZ basis set, then that is the calculation that may be launched. The energies, forces, atomic numbers, and atomic coordinates from this calculation may be saved for further training of the ML potential.

In some cases, the method 100, 200, 300, or 400 further comprises, until a completion criterion is met: (i) if the reliability metric is less than the threshold reliability value: evaluating the energy or the force at the coordinate that was unreliable, selecting another coordinate on the energy surface, using the trained model to evaluate the energy or the force at the another coordinate, and determining a reliability metric for the energy or the force at the another coordinate; and (ii) if the reliability metric is greater than the threshold reliability value: selecting another coordinate on the energy surface, using the trained model to evaluate the energy at the another coordinate, and determining a reliability metric for the energy or the force at the another coordinate.

In some cases, at (i), the method further comprises saving the energy or force for retraining and retraining the trained model based on the energy or the force in (i). In some embodiments, selecting another coordinate on the energy surface comprises a method selected from the group consisting of: ab initio molecular dynamics, nudged elastic band, growing string method, a variational reaction path optimization method, and intrinsic reaction coordinates.

Referring back to FIG. 4, in some cases, the method may comprise an operation 425: if a reliability metric is less than the threshold reliability value: evaluating an ab initio energy or an ab initio force at the conformational coordinate. After operation 425, the method may comprise: selecting another conformation coordinate on the potential energy surface; using a trained model to evaluate the energy or the force at another conformational coordinate; and determining the reliability metric for the energy or the force at another conformational coordinate.

In some cases, the method may comprise an operation 430: if the reliability metric is greater than the threshold reliability value: selecting another conformational coordinate on the potential energy surface. After operation 425, the method may comprise using the trained model to evaluate the energy at another conformational coordinate and determining a reliability metric for the energy or the force at another conformational coordinate.

In some cases, the method may comprise an operation 440 determining whether a completion condition is met. If the completion condition is not met, the method may comprise repeating operations 410, 420, 425, and 430 until the completion condition is met.

Reliability Metric—In some cases, the method 100, 200, 300, or 400 further comprises determining a reliability metric. In some cases, a computational chemistry model can output its own reliability metric. For example, if the computational chemistry method outputs an error assessment, then the error estimate may be used as a reliability metric. A Bayesian model may be an example of a model that produces a measure of uncertainty. For example, gaussian process regression may produce a measure of uncertainty. In some cases, if a reaction path calculated is not smooth, then there may be a higher uncertainty of the value that is significantly off of a smooth curve. In some cases, the same ML model may be implemented a plurality of times and a standard deviation may be calculated.

In some cases, a reliability test includes standard deviation computation using the inference outcomes based on plurality of ML models. In some cases, the plurality of ML models is part of an ensemble learning model. In some cases, the reliability test includes the similarity search between the training dataset and the input query to identify its rareness. In some cases, the inference reliability test includes instance selection strategies for selecting critical instances to refine the ML models. Instance selection strategies include the uncertainty sampling approach, the query by committee approach, the expected model change approach, the expected error reduction approach, and the density weighted methods.

In some cases, determining a reliability metric comprises combining results from one or more sub-models to create a metamodel, calculating an energy for each of the one or more sub-models, and calculating a standard deviation of energy for the one or more sub-models. In some cases, the standard deviation comprises a part of the reliability metric. For example, one can estimate the reliability of a given evaluation by taking the standard deviation of the energies predicted by the sub-models of an ensemble learning method. In some cases, the standard deviation may be normalized based on the model architecture in order to produce a consistent estimate for the reliability of a model. For example, in the ensemble ANI architecture, the normalized standard deviation is given by:

$$ρ = \frac{σ}{\sqrt{N}}$$

where σ is the standard deviation in the usual sense, and N is the number of atoms in the molecule.

### Quantum Chemistry Calculation

At an operation 150, in response to the reliability metric the method may comprise, optionally, evaluating an energy or a force based on a quantum chemistry calculation. The quantum chemistry calculation may correspond to a training data set of said trained model. For example, the type of quantum chemistry calculation launched may match that used to generate the original training dataset of the ML potential. For example, if the original training set was calculated using coupled cluster singles and doubles with the cc-pVTZ basis set, then that is the calculation that may be launched. The energies, forces, atomic numbers, and atomic coordinates from this calculation may be saved for further training of the ML potential.

For example, if the ML potential is not reliable enough, a quantum chemistry calculation may be launched to calculate energies and forces ab initio. The type of quantum chemistry calculation launched may match that used to generate the original training dataset of the ML potential. For example, if the original training set was calculated using coupled cluster singles and doubles with the cc-pVTZ basis set, then that is the calculation that may be launched. The energies, forces, atomic numbers, and atomic coordinates from this calculation may be saved for further training of the ML potential.

In some cases, evaluating an energy or a force based on a quantum chemistry calculation comprises evaluating an ab initio energy or an ab initio force at the coordinate. In some cases, the ab initio energy or the ab initio force is calculated by a Hartree-Fock method, a coupled cluster method, full configuration interaction, incremental full configuration interaction, density functional theory, Moller-Plesset perturbation theory, mixed quantum mechanical and molecular mechanical methods, density matrix embedding theory, or ONIOM models.

In some cases, computing the energy or the force comprises at least one member of the group consisting of Hartree-Fock (HF) method, Density Functional Theory (DFT), Coupled-Cluster Single-, Double-, and perturbative Triple-excitations (CCSD (T)), Full Configuration Interaction (FCI), Heat-Bath Configuration Interaction (HBCI), Quantum Monte Carlo Full Configuration Interaction (QMCFCI), Density Matrix Embedding Theory (DMET), Fragment Molecular Orbital method (FMO), Incremental Full Configuration Interaction (iFCI), Hybrid quantum mechanics-molecular mechanics (QM/MM), and Ab initio molecular dynamics (AIMD) simulation, Variational Monte Carlo, and Diffusion Monte Carlo.

In some cases, the method 100, 200, 300, or 400 further comprises saving the energy or the force from a quantum chemistry calculation for retraining the trained model.

In some cases, the method 100, 200, 300, or 400 further comprises, after calculating the force or the energy with a higher level of theory, selecting another conformation coordinate on the potential energy surface. At the next coordinate, the method may comprise using a trained model to evaluate the energy or the force at the new conformational coordinate. At the new coordinate, the method may comprise determining the reliability metric for the energy or the force at the new conformational coordinate.

### Model Outputs

In some cases, the method 100, 200, 300, or 400 further comprises if the reliability metric is greater than the threshold reliability value: selecting another conformational coordinate on the potential energy surface. The method may comprise using the trained model to evaluate the energy at another conformational coordinate and determining a reliability metric for the energy or the force at another conformational coordinate.

In some cases, the method 100, 200, 300, or 400 further comprises determining whether a completion condition is met. If the completion condition is not met, the method may comprise repeating operations 410, 420, 425, and 430 until the completion condition is met. The completion condition may comprise a reaching a threshold value, such as a maximum number of cycles, a threshold change in the energy, a threshold change in a reliability metric, etc.

In some cases, the method 100, 200, 300, or 400 further comprises outputting a transition state or a reaction path on the energy surface. In some cases, the reaction path is a minimum energy path. In some cases, the method 100, 200, 300, or 400 further comprises: outputting a set of energies or forces at the set of coordinates on the energy surface based at least in part on the energy or force calculated from the higher level of theory method (e.g., a quantum chemistry method).

In some cases, the method 100, 200, 300, or 400 further comprises outputting a set of energies or forces at the set of conformational coordinates on the potential energy surface. In some cases, the outputted set of energies or forces are output after a completion condition is met.

### ML Retraining

Methods and systems of the present disclosure may also be integrated with retraining or updating the trained model. For example, the present application provides methods and systems for determining a reaction path comprising retraining the trained model. Referring again to FIG. 3, there is a flowchart of an example of a method 300 for determining a reaction path and using data from a quantum chemistry calculation to update a trained model. At an operation 301, method 300 may comprise providing an indication of a reactant and at least one of a product or a driving coordinate. At an operation 310, method 300 may comprise providing a set of coordinates. The set of coordinates may be on an energy surface connecting the reactant and at least one of the product or the driving coordinate. At an operation 320, method 300 may comprise evaluating an energy or a force at a coordinate of the set of coordinates using a trained model. At an operation 330, method 300 may comprise determining that a reliability metric at the coordinate is less than a threshold reliability value. At an operation 340, method 300 may comprise evaluating an energy or a force at the coordinate based at least in part on a quantum chemistry calculation corresponding to a training data set of the trained model. At an operation 350, method 300 may comprise retraining the trained model based on the energy or the force in 340.

In an example of the method 300, the present application provides a computer-implemented method for determining a reaction path. The method may comprise: (a) providing an indication of a reactant and at least one of a product or a driving coordinate; (b) providing a set of conformational coordinates, wherein the set of conformational coordinates is on a potential energy surface connecting the reactant and the at least one of the product or the driving coordinate; (c) using a trained model to evaluate an energy or a force at a conformational coordinate of the set of conformational coordinates; (d) evaluating an ab initio energy or an ab initio force at the conformational coordinate; and (e) retraining the trained model based on the ab initio energy or the ab initio force.

In some cases, the method 100, 200, 300, or 400 further comprises passing energies and forces based on the training data to machine learning model. In some cases, the energies and forces from a model corresponding to the training data may be passed back to the reaction path prediction algorithm. In some cases, the energies and forces from the quantum chemistry calculation may be passed back to the machine learning model. In some cases, the energies and forces from the quantum chemistry calculation may be passed back to the reaction path prediction algorithm. Once the reaction path prediction algorithm receives these quantities, it may continue. In some cases, it may continue with no other modifications. Retraining of the ML potential to incorporate the new data points generated above may occur separately to the reaction path prediction process. This retraining does not need to occur during the reaction path prediction process, although it may.

### Computer Systems

Systems and methods of the present disclosure may implement various operations on a digital computer. In some cases, a digital computer comprises one or more hardware central processing units (CPUs) that carry out the digital computer's functions. In some cases, the digital computer further comprises an operating system configured to perform executable instructions. In some cases, the digital computer is connected to a computer network. In some cases, the digital computer is connected to the Internet such that it accesses the World Wide Web. In some cases, the digital computer is connected to a cloud computing infrastructure. In some cases, the digital computer is connected to an intranet. In some cases, the digital computer is connected to a data storage device.

Various types of digital computer may be used. In fact, suitable digital computers may include, by way of non-limiting examples, server computers, desktop computers, laptop computers, notebook computers, sub-notebook computers, netbook computers, netbook computers, set-top computers, media streaming devices, handheld computers, Internet appliances, mobile smartphones, tablet computers, personal digital assistants, video game consoles, and vehicles. Smartphones may be suitable for use with one or more examples of the method and the system described herein. Select televisions, video players, and digital music players, in some cases with computer network connectivity, may be suitable for use in some cases of the system and the method described herein. Suitable tablet computers may include those with booklet, slate, and convertible configurations.

In some cases, the digital computer comprises an operating system configured to perform executable instructions. The operating system may be, for example, software, comprising programs and data, which manages the device's hardware and provides services for execution of applications. Various types of operating system may be used. For example, suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Suitable personal computer operating systems may include, by way of non-limiting examples, Microsoft® Windows®, Apple® Mac OS X®, UNIX®, and UNIX-like operating systems such as GNU/Linux®. In some cases, the operating system is provided by cloud computing. Suitable mobile smart phone operating systems may include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Suitable media streaming device operating systems may include, by way of non-limiting examples, Apple TV®, Roku®, Boxee®, Google TV®, Google Chromecast®, Amazon Fire®, and Samsung® HomeSync®. Suitable video game console operating systems may include, by way of non-limiting examples, Sony® PS3®, Sony® PS4R, Microsoft® Xbox 360®, Microsoft® Xbox One®, Nintendo® Wii®, Nintendo® Wii U®, and Ouya®.

In some cases, the digital computer comprises a storage and/or memory device. Various types of storage and/or memory may be used in the digital computer. In some cases, the storage and/or memory device comprises one or more physical apparatuses used to store data or programs on a temporary or permanent basis. In some cases, the device comprises a volatile memory and requires power to maintain stored information. In some cases, the device comprises non-volatile memory and retains stored information when the digital computer is not powered. In some cases, the non-volatile memory comprises a flash memory. In some cases, the non-volatile memory comprises a dynamic random-access memory (DRAM). In some cases, the non-volatile memory comprises a ferroelectric random-access memory (FRAM). In some cases, the non-volatile memory comprises a phase-change random access memory (PRAM). In some cases, the device comprises a storage device including, by way of non-limiting examples, CD-ROMs, DVDs, flash memory devices, magnetic disk drives, magnetic tapes drives, optical disk drives, and cloud computing-based storage. In some cases, the storage and/or memory device comprises a combination of devices, such as those disclosed herein.

In some cases, the digital computer comprises a display used for providing visual information to a user. Various types of display may be used. In some cases, the display comprises a cathode ray tube (CRT). In some cases, the display comprises a liquid crystal display (LCD). In some cases, the display comprises a thin film transistor liquid crystal display (TFT-LCD). In some cases, the display comprises an organic light-emitting diode (OLED) display. In some cases, an OLED display comprises a passive-matrix OLED (PMOLED) or active-matrix OLED (AMOLED) display. In some cases, the display comprises a plasma display. In some cases, the display comprises a video projector. In some cases, the display comprises a combination of devices, such as those disclosed herein.

In some cases, the digital computer comprises an input device to receive information from a user. Various types of input devices may be used. In some cases, the input device comprises a keyboard. In some cases, the input device comprises a pointing device including, by way of non-limiting examples, a mouse, trackball, trackpad, joystick, game controller, or stylus. In some cases, the input device comprises a touch screen or a multi-touch screen. In some cases, the input device comprises a microphone to capture voice or other sound input. In some cases, the input device comprises a video camera or other sensor to capture motion or visual input. In some cases, the input device comprises a Kinect™, Leap Motion™, or the like. In some cases, the input device comprises a combination of devices, such as those disclosed herein.

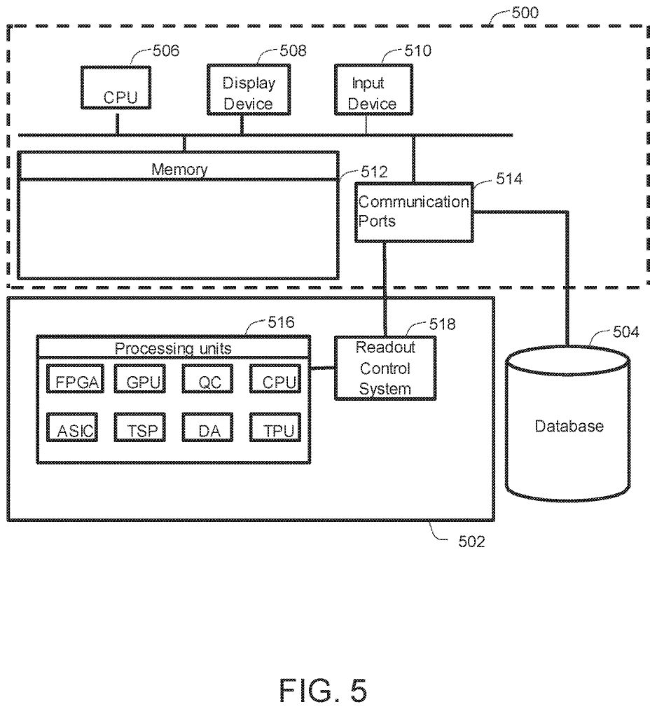

Now referring to FIG. 5, there is shown an example schematic diagram of a system for determining a reaction path. The system is configured to determine a reaction path utilizing one or more machine learning (ML) models, e.g., a neural network, an ensemble machine learning model, etc.

The system may comprise a digital computer 500. The digital computer may be a digital computer of various types, such as, for example, a digital computer as described elsewhere herein. The digital computer may comprise at least one processing device 506 and at least one memory 512. The at least one memory may comprise a computer program executable by the processing device 506 which may be configured to provide or receive an indication of a reactant and a product or a driving coordinate; provide or receive a set of coordinates on an energy surface; evaluate an energy or a force using a trained model; determine a reliability metric for the energy or the force at the coordinate determined with the trained model; and, in response to the reliability metric, optionally, evaluate an energy or a force based on a quantum chemistry calculation.

The system may comprise a computational platform 502 operatively connected to the digital computer 500. The computational platform 502 may comprise at least one processor 516. The at least one processor 516 may be of various types of processors such as, for example, the types of processors as described elsewhere herein. The at least one processor can include Noisy Intermediate-Scale Quantum (NISQ) technology, any quantum device, any high-performance computing device any quantum annealer, any optical computing device, an integrated photonic coherent Ising machine etc. For example, the at least one processor can comprise at least one field-programmable gate array (FPGA), application-specific integrated circuit (ASIC), central processing unit (CPU), graphics processing unit (GPU), tensor processing unit (TPU), tensor streaming processor (TSP), quantum computer, quantum annealer, integrated photonic coherent Ising machine, optical quantum computer, or the like, or any combination thereof. The computational platform 502 may be provided by a cloud computing system. In some cases, computational platform 502 comprises an array of distributed high performance computing units. The distributed high performance computing units may comprise processing units of various types as described herein.

Each component of the system (e.g., the hardware) may be used as part of the system to execute a whole method, or any portion thereof, alone or in combination with other components (e.g., other hardware). In some cases, the components may be used for obtaining a request comprising an indication of at least one property of a molecule and a task, performing inference on at least one machine learning (ML) model using the obtained indication, performing inference reliability test, if the reliability is satisfactory obtaining task result using the inference outcomes, if the reliability is not satisfactory, performing the task to obtain task result.

The computational platform 502 may be operatively connected to the digital computer 500. The computational platform may be communicatively coupled to the digital computer. The computational platform may comprise a read-out control system 518. The read-out control system may be configured to read information (e.g., computational results, parameters, etc.) from the at least one processor 516. For example, the read-out control system can be configured to convert data from an FPGA to data usable by a digital computer.

The system may comprise a database 504. The database 504 may be operatively connected to the digital computer 500. The database 504 may be a database of various types. The database 504 may refer to a central repository configured to save the specification of the task and task results. In some cases, the database can be, for example, MongoDB. The database 504 may be used to store indications of properties of molecules, corresponding tasks and results thereof. The database 504 may be used to store the task results. The database 504 may be further used to store the output from chemistry discovery toolbox. The database 504 may be further used to store the dataset for training the ML models. The dataset for training ML models may be a subset or complete set of task results. The dataset for training ML models may further be a subset or complete set of the output from chemistry discovery toolbox. The processing device 506 may be further configured to store in the database 504 indications of properties of molecules, corresponding tasks and the results thereof and to read from the database 504 indications of properties of molecules.

The computational platform 502 and the database 504 may be connected to the digital computer 500 over a network. The computational platform, the database, and/or the digital computer can have network communication devices. The network communication devices can enable the computational platform, the database, and/or the digital computer to communicate with each other and with any number of user devices, over a network. The network can be a wired or wireless network. For example, the network can be a fiber optic network, Ethernet® network, a satellite network, a cellular network, a Wi-Fi® network, a Bluetooth® network, or the like. In one or more implementations, the computational platform, the database, and/or digital computer can be several distributed computational platforms, databases, and/or the digital computers that are accessible through the Internet. Such computational platforms, databases, and/or digital computers may be considered cloud computing devices. In some cases, the one or more processors of the at least one processor may be located in the cloud.

The at least one processor 516 may comprise one or more virtual machines. The one or more virtual machines may be one or more emulations of one or more computer systems. The virtual machines may be process virtual machines (e.g., virtual machines configured to implement a process in a platform-independent environment). The virtual machines may be systems virtual machines (e.g., virtual machines configured to execute an operating system and related programs). The virtual machine may be configured to emulate a different architecture from the at least one processor. For example, the virtual machine may be configured to emulate a quantum computing architecture on a silicon computer chip. Examples of virtual machines may include, but are not limited to, VMware®, VirtualBox®, Parallels®, QEMU®, Citrix® Hypervisor, Microsoft® Hyper-V®, or the like.

The system of FIG. 5 may provide or receive an indication of a reactant and at least one of a product or a driving coordinate; provide or receive a set of conformational coordinates, wherein the set of conformational coordinates is on a potential energy surface connecting the reactant and the at least one of the product or the driving coordinate; use a trained model to evaluate an energy or a force at a conformational coordinate of the set of conformation coordinates; determine a reliability metric at the conformational coordinate; and, optionally, evaluate an ab initio energy or an ab initio force at the conformational coordinate.

In some cases, at an input device 516, digital computer 500 may receive a set of conformational coordinates, an indication of a reactant, at least one of a product or a driving coordinate, or any combination thereof. In some cases, at a processor 506, the system may, based on an indication from a user, provide a set of conformational coordinates, an indication of a reactant, at least one of a product or a driving coordinate, or any combination thereof. In some cases, digital computer 500 is a device of a user providing a user access to an application programming interface.

In some cases, the system is configured to use a trained model to evaluate an energy or a force at a conformational coordinate of the set of conformation coordinates. In some cases, the system is configured direct a processing unit 516 to do any of the following: use a trained model to evaluate an energy or a force at a conformational coordinate of the set of conformation coordinates; determine a reliability metric at the conformational coordinate; evaluate an ab initio energy or an ab initio force at the conformational coordinate; or any combination thereof. The system may implement the trained model over a communication port. In some cases, the system is configured direct a CPU 506 to do any of the following: use a trained model to evaluate an energy or a force at a conformational coordinate of the set of conformation coordinates; determine a reliability metric at the conformational coordinate; evaluate an ab initio energy or an ab initio force at the conformational coordinate; or any combination thereof. In some cases, processing unit 516 comprises an array of distributed high performance computing units. The distributed high performance computing units may comprise processing units of various types as described herein.

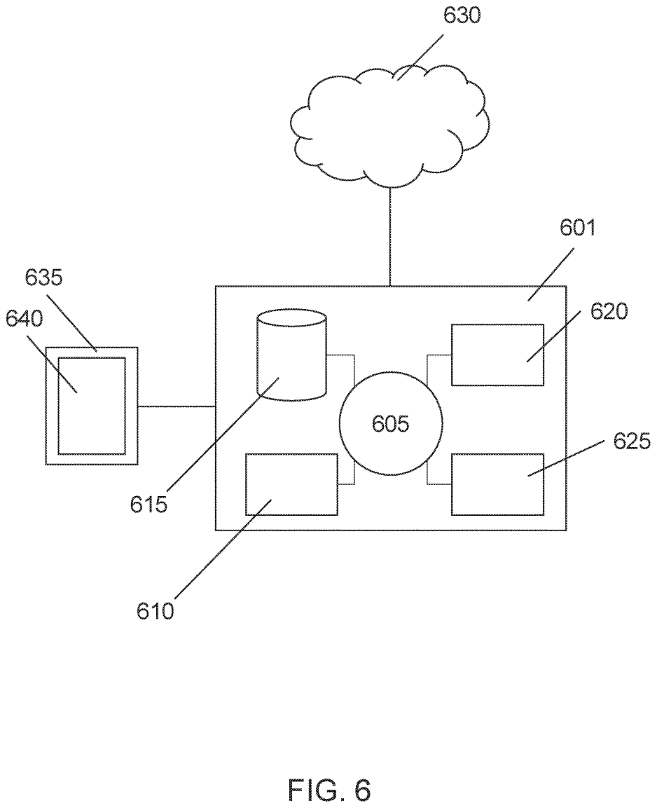

The present disclosure provides computer systems that are programmed to implement methods of the disclosure. FIG. 6 shows a computer system 601 that is programmed or otherwise configured to perform operations of the methods for determining a reaction path disclosed herein. Computer system 601 may comprise an embodiment variation or example of a digital computer 500 of FIG. 5. For example, computer system 601 may be programmed or otherwise configured to (a) provide or receive an indication of a reactant and at least one of a product or a driving coordinate; (b) provide or receive a set of conformational coordinates, wherein the set of conformational coordinates is on a potential energy surface connecting the reactant and the at least one of the product or the driving coordinate; (c) use a trained model to evaluate an energy or a force at a conformational coordinate of the set of conformation coordinates; (d) determine that a reliability metric at the conformational coordinate is less than a threshold reliability value; (e) evaluate an ab initio energy or an ab initio force at the conformational coordinate; and (f) output a set of energies or forces at the set of conformational coordinates on the potential energy surface based at least in part on the ab initio energy or ab initio force and the energy or the force.

The computer system 601 can be an electronic device of a user or a computer system that is remotely located with respect to the electronic device. The electronic device can be a mobile electronic device.

The computer system 601 includes a central processing unit (CPU, e.g., CPU 506, also “processor” and “computer processor” herein) 605, which can be a single core or multi core processor, or a plurality of processors for parallel processing. The computer system 601 also includes memory or memory location 610 (e.g., random-access memory, read-only memory, flash memory, e.g., memory 512), electronic storage unit 615 (e.g., hard disk), communication interface 620 (e.g., network adapter, e.g., communication port 514) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine-readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 (e.g., display device 508) that comprises a user interface (UI) 640 (e.g., input device 510) for providing, for example, an output of coordinates, structures, energies, geometries, etc. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 605. The algorithm can, for example, execute one or more operations of the methods for determining a reaction path disclosed herein.

### Quantum Computer System

In some cases, the systems and methods disclosed herein may be performed with the aid of a quantum computing system. In some cases, a computer-implemented method of the present disclosure may be performed at least partially by a quantum computer. In some cases, a computing system of the present disclosure may comprise a hybrid computing unit. In some cases, a hybrid computing unit may comprise a classical computer and quantum computer. The quantum computer may be configured to perform one or more quantum algorithms for solving a computational problem (e.g., at least a portion of a reaction path calculation). A quantum processor may comprise processing unit QC of element 516 of FIG. 5.

The one or more quantum algorithms may be executed using a quantum computer, a quantum-ready computing service, or a quantum-enabled computing service. For instance, the one or more quantum algorithms may be executed using the systems or methods described in U.S. Patent Publication No. 2018/0107526, entitled “METHODS AND SYSTEMS FOR QUANTUM READY AND QUANTUM ENABLED COMPUTATIONS”, which is entirely incorporated herein by reference. The classical computer may comprise at least one classical processor and computer memory and may be configured to perform one or more classical algorithms for solving a computational problem (e.g., at least a portion of a reaction path calculation).

The digital computer may comprise at least one computer processor and computer memory, wherein the digital computer may include a computer program with instructions executable by the at least one computer processor to render an application. The application may facilitate use of the quantum computer and/or the classical computer by a user.

Some implementations may use quantum computers along with classical computers operating on bits, such as personal desktops, laptops, supercomputers, distributed computing, clusters, cloud-based computing resources, smartphones, or tablets.

The system may comprise an interface for a user. In some cases, the interface may comprise an application programming interface (API). The interface may provide a programmatic model that abstracts away (e.g., by hiding from the user) the internal details (e.g., architecture and operations) of the quantum computer. In some cases, the interface may minimize a need to update the application programs in response to changing quantum hardware. In some cases, the interface may remain unchanged when the quantum computer has a change in internal structure.

The present disclosure provides systems and methods that may include non-classical (e.g., quantum) computing or use of non-classical (e.g., quantum) computing. Quantum computers may be able to solve certain classes of computational tasks more efficiently than classical computers. However, quantum computation resources may be rare and expensive, and may involve a certain level of expertise to be used efficiently or effectively (e.g., cost-efficiently or cost-effectively). A number of parameters may be tuned in order for a quantum computer to deliver its potential computational power.

Quantum computers (or other types of non-classical computers) may be able to work alongside classical computers as co-processors. A hybrid architecture (e.g., computing system) comprising a classical computer and a quantum computer can be very efficient for addressing complex computational tasks, such as quantum chemistry simulations. Systems and methods disclosed herein may be able to efficiently and accurately decompose or break down a quantum chemistry problem and delegate appropriate components of the quantum chemistry simulations to the quantum computer or the classical computer.

Although the present disclosure has referred to quantum computers, methods and systems of the present disclosure may be employed for use with other types of computers, which may be non-classical computers. Such non-classical computers may comprise quantum computers, hybrid quantum computers, quantum-type computers, or other computers that are not classical computers. Examples of non-classical computers may include, but are not limited to, Hitachi Ising solvers, coherent Ising machines based on optical parameters, and other solvers which utilize different physical phenomena to obtain more efficiency in solving particular classes of problems.

In some cases, a quantum computer may comprise one or more adiabatic quantum computers, quantum gate arrays, one-way quantum computers, topological quantum computers, quantum Turing machines, superconductor-based quantum computers, trapped ion quantum computers, trapped atom quantum computers, optical lattices, quantum dot computers, spin-based quantum computers, spatial-based quantum computers, Loss-DiVincenzo quantum computers, nuclear magnetic resonance (NMR) based quantum computers, solution-state NMR quantum computers, solid-state NMR quantum computers, solid-state NMR Kane quantum computers, electrons-on-helium quantum computers, cavity-quantum-electrodynamics based quantum computers, molecular magnet quantum computers, fullerene-based quantum computers, linear optical quantum computers, diamond-based quantum computers, nitrogen vacancy (NV) diamond-based quantum computers, Bose-Einstein condensate-based quantum computers, transistor-based quantum computers, and rare-earth-metal-ion-doped inorganic crystal based quantum computers. A quantum computer may comprise one or more of: quantum annealers, Ising solvers, optical parametric oscillators (OPO), and gate models of quantum computing.

In some cases, a non-classical computer of the present disclosure may comprise a noisy intermediate-scale quantum device. “Noisy” may imply that incomplete control over the qubits is present and the “Intermediate-Scale” may refer to the number of qubits which may range from 50 to a few hundreds. Several physical systems made from superconducting qubits, artificial atoms, ion traps are proposed so far as feasible candidates to build NISQ quantum device and ultimately universal quantum computers.

In some cases, a classical simulator of the quantum circuit can be used which can run on a classical computer like a MacBook Pro laptop, a Windows laptop, or a Linux laptop. In some cases, the classical simulator can run on a cloud computing platform having access to multiple computing nodes in a parallel or distributed manner. In some cases, all or a portion of a quantum mechanical energy and/or electronic structure calculation may be performed using the classical simulator.

The methods described herein may be performed on an analogue quantum simulator. An analogue quantum simulator may be a quantum mechanical system consisting of a plurality of manufactured qubits. An analogue quantum simulator may be designed to simulate quantum systems by using physically different but mathematically equivalent or approximately equivalent systems. In an analogue quantum simulator, each qubit may be realized in an ion of strings of trapped atomic ions in linear radiofrequency traps. To each qubit may be coupled a source of bias called a local field bias. The local field biases on the qubits may be programmable and controllable. In some cases, a qubit control system comprising a digital processing unit is connected to the system of qubits and is capable of programming and tuning the local field biases on the qubits.

### EXAMPLES

### Example 1: Diels Alder GSM Experiment

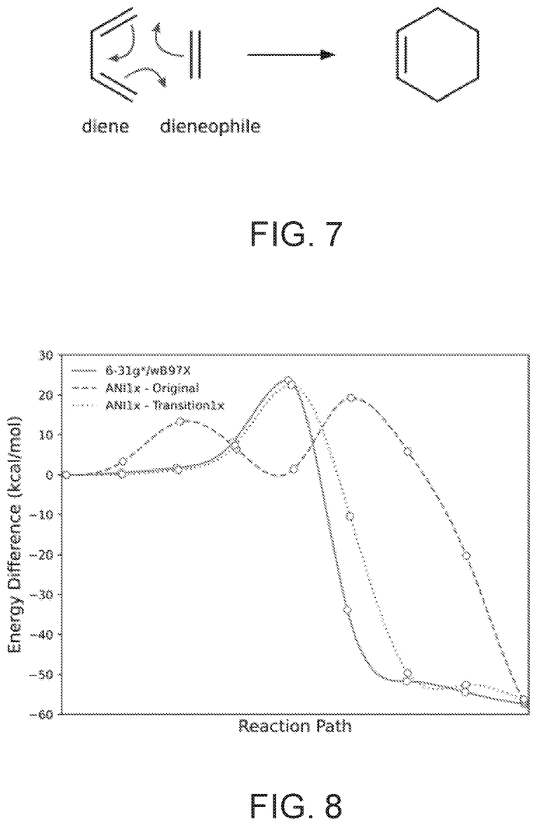

FIG. 7 is a diagram of a chemical reaction between s-cis-butadiene and ethene to form cyclohexene. The Growing String Method (GSM) was used to predict the transition state and reaction path coming from the s-cis-butadiene+ethene→cyclohexene reaction, shown in FIG. 7. This reaction path was predicted using several different models to evaluate the efficacy of the ML model with different training data. It was found that this experiment was a proof-of-concept that ML models can effectively model transition states and reaction paths when retrained on relevant data. It has been shown that when an ensemble ML model has high variability, running a quantum chemistry calculation and retraining the ML model on that data, the ML model will have a high chance of success at reaction path prediction.

Methods: The following energy and force evaluators were used: Density functional theory (DFT) at the 6-31G*/ωB97X level of theory. The ANI-1x machine learning (ML) model trained on the original ANI-1x dataset, referred to as “ANI/ANI1x.” The ANI-1x ML model trained on the Transition-1x dataset, referred to as “ANI/Transition1x.” For each evaluator, the reactant and product structures were optimized to local minima prior to the GSM calculation. GSM calculations were then performed using the corresponding evaluator to calculate the energy and forces of each GSM node during the string growing and optimization process.

Results: Reaction path prediction algorithms can be evaluated both on the quality of the transition state they predict and the overall quality of the reaction path that is calculated. In this case, the ANI/ANI1x evaluator both performed worse quantitatively than the ANI/Transition1x evaluator and failed to qualitatively predict the correct transition state.

To compare these models quantitatively, the absolute energies and reaction barriers of each ML evaluator are compared to the DFT evaluator. (See Table 1 for values) The deviation of the ANI/ANI1x evaluator from the DFT results is approximately 0.0117 Hartree and 4.4 kcal/mol for the absolute energy and reaction barrier, respectively. The deviation of the ANI/Transition1x evaluator from the DFT results is approximately 0.0040 Hartree and 1.2 kcal/mol, respectively. Not only does the ANI/Transition1x evaluator outperform the ANI/ANI1x evaluator, but it also predicts a reaction barrier to nearly within chemical accuracy (1 kcal/mol) of the DFT result.

FIG. 8 is a plot of computational data showing: reaction path energy profiles for DFT (solid), ANI/ANI1x (dashed), and ANI/Transition1x (dotted); GSM node energies (open circles); and intermediate values estimated with a cubic spline interpolation (regression lines). Qualitatively, it can be seen that the ANI/ANI1x evaluator predicts a different reaction mechanism than both DFT and ANI/Transition1x. DFT and ANI/Transition1x predict the correct transition state where the ethene approaches the butadiene from outside the butadiene bonding plane, ANI/ANI1x predicts a transition state that involves torsion of the C2-C3 bond and direct attachment of the ethene across the bonding plane of the butadiene. This can be seen in the reaction path where the double-barrier nature of the ANI/ANI1x reaction path comes from butadiene torsion first and bond formation second.

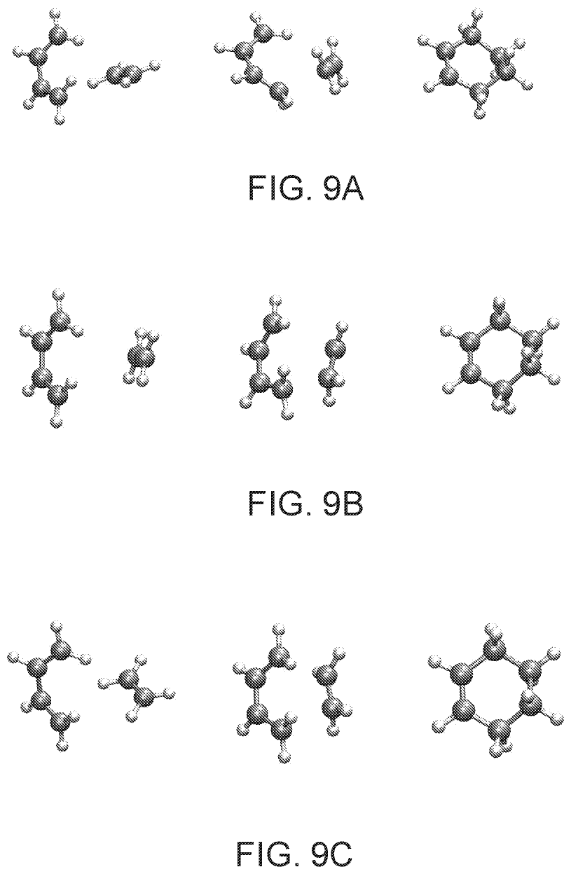

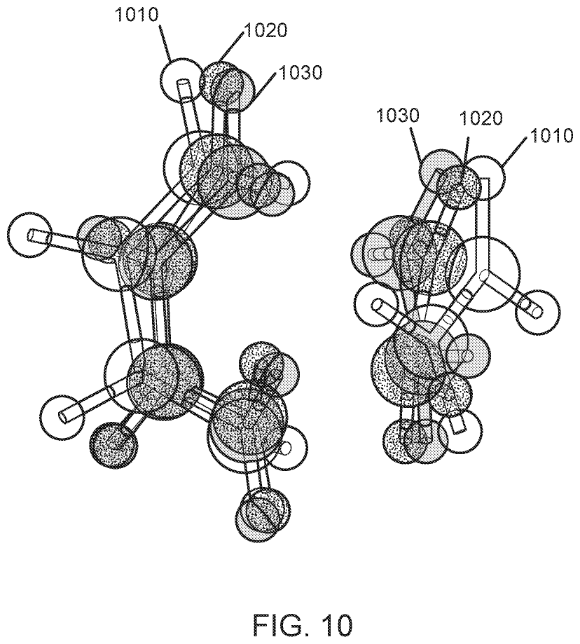

FIG. 9A is computational data showing molecular structures for the reactants (left), transition state (center), and products (right) predicted by GSM using the ANI/ANI1x model. FIG. 9B is computational data showing molecular structures for the reactants (left), transition state (center), and products (right) predicted by GSM using the ANI/Transition1x model. FIG. 9C is computational data showing molecular structures for the reactants (left), transition state (center), and products (right) predicted by GSM using the DFT model. As shown from the snapshots of the transition state geometries (center) in FIG. 9A-9C, the ANI/ANI1x evaluator predicts a different reaction mechanism than both DFT and ANI/Transition1x. DFT and ANI/Transition1x predict the correct transition state where the ethene approaches the butadiene from outside the butadiene bonding plane, ANI/ANI1x predicts a transition state that involves torsion of the C2-C3 bond and direct attachment of the ethene across the bonding plane of the butadiene

FIG. 10 shows overlaid transition state structures predicted by GSM using DFT, (filled, 1030) ANI/ANI1x, (open, 1010) and ANI/Transition1x. (textured, 1020), where all structures have been translated to have the same overall center of mass. As shown, FIG. 10 also indicates differences in transition state structures.

TABLE 1
Absolute energy of the transition state and reaction barrier
predicted by GSM using each energy and force evaluator.

| | Absolute energy | Reaction barrier |
|---|---|---|
| Evaluator | (Hartree) | (kcal/mol) |
| DFT | −234.45489899 | 23.7 |
| ANI/ANI1x | −234.46654967 | 19.3 |
| ANI/Transition1x | −234.45889929 | 22.5 |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1. A computer-implemented method for determining a reaction path, said method comprising:
(a) providing an indication of a reactant and at least one of a product or a driving coordinate;
(b) providing a set of coordinates, wherein said set of coordinates is on an energy surface connecting said reactant and said at least one of said product or said driving coordinate;
(c) evaluating an energy or a force at a coordinate of said set of coordinates using a trained model;
(d) determining that a reliability metric at said coordinate is less than a threshold reliability value;
(e) evaluating an energy or a force at said coordinate based at least in part on a quantum chemistry calculation corresponding to a training data set of said trained model; and
(f) outputting a set of energies or forces at said set of coordinates on said energy surface based at least in part on said energy or force in (e) and said energy or said force in (c).

2. The computer-implemented method of claim 1, wherein said energy surface is a potential energy surface.

3. The computer-implemented method of claim 1, wherein said energy surface is a free energy surface.

4. The computer-implemented method of claim 1, wherein said set of coordinates are a conformation coordinate.

5. The computer-implemented method of claim 1, wherein said set of coordinates are cartesian coordinates and said cartesian coordinates comprise a direction of movement.

6. (canceled)

7. The computer-implemented method of claim 1, wherein (e) comprises evaluating an ab initio energy or an ab initio force at said coordinate.

8. The computer-implemented method of claim 1, further comprising: until a completion criterion is met:
(i) if said reliability metric is less than said threshold reliability value:
evaluating said energy or said force at said coordinate in (c), selecting another coordinate on said energy surface, using said trained model to evaluate said energy or said force at said another coordinate, and determining a reliability metric for said energy or said force at said another coordinate; and
(ii) if said reliability metric is greater than said threshold reliability value:
selecting another coordinate on said energy surface, using said trained model to evaluate said energy at said another coordinate, and determining a reliability metric for said energy or said force at said another coordinate.

9. The computer-implemented method of claim 1, further comprising at (e) saving said energy or said force for retraining and retraining said trained model based on said energy or said force in (c).

10. The computer-implemented method of claim 8, further comprising at (i) saving said energy or force for retraining and retraining said trained model based on said energy or said force in (i).

11. The computer-implemented method of claim 1, further comprising using said trained model to evaluate a first energy or a first force at an initial coordinate and determining a reliability metric for said first energy or said first force at said initial coordinate.

12. (canceled)

13. The computer-implemented method of claim 1, wherein (f) further comprises outputting a transition state or a reaction path on said energy surface, wherein said reaction path is a minimum energy path.

14. (canceled)

15. The computer-implemented method of claim 1, wherein said providing a set of coordinates in (b) comprises a method selected from the group consisting of: ab initio molecular dynamics, nudged elastic band, growing string method, a variational reaction path optimization method, and intrinsic reaction coordinate.

16. The computer-implemented method of claim 8, wherein said selecting said another coordinate on said energy surface comprises a method selected from the group consisting of: ab initio molecular dynamics, nudged elastic band, growing string method, a variational reaction path optimization method, and intrinsic reaction coordinate.

17. The computer-implemented method of claim 1, wherein said trained model comprises a machine learning algorithm, wherein said machine learning algorithm comprises a neural network or an ensemble learning method.

18. (canceled)

19. (canceled)

20. The computer-implemented method of claim 17, wherein (d) comprises: combining results from one or more sub-models to create a metamodel, calculating an energy for each of said one or more sub-models, and calculating a standard deviation of energy for said one or more sub-models, wherein said standard deviation comprises a part of said reliability metric.

21. The computer-implemented method of claim 20, wherein said metamodel comprises an ANI deep learning potential.

22. The computer-implemented method of claim 20, wherein said one or more sub-models comprises one or more of PAINN deep learning potentials, DimeNet++ deep learning potentials, or PauliNet.

23. The computer-implemented method of claim 1, wherein (e) comprises evaluating an ab initio energy or an ab initio force at said coordinate, and wherein said ab initio energy or said ab initio force is calculated by a Hartree-Fock method, a coupled cluster method, full configuration interaction, incremental full configuration interaction, density functional theory, Moller-Plesset perturbation theory, mixed quantum mechanical and molecular mechanical methods, density matrix embedding theory, or ONIOM models.

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. A processor comprising a non-transitory medium with instructions stored thereon which when executed instruction one or more processing units to perform operations for determining a reaction path, said operations comprising:
(a) providing an indication of a reactant and at least one of a product or a driving coordinate;
(b) providing a set of coordinates, wherein said set of coordinates is on an energy surface connecting said reactant and said at least one of said product or said driving coordinate;
(c) evaluating an energy or a force at a coordinate of said set of coordinates using a trained model;
(d) determining that a reliability metric at said coordinate is less than a threshold reliability value;
(e) evaluating an energy or a force at said coordinate based at least in part on a quantum chemistry calculation corresponding to a training data set of said trained model; and
(f) outputting a set of energies or forces at said set of coordinates on said energy surface based at least in part on said energy or force in (e) and said energy or said force in (c).

67. The processor of claim 66, wherein said one or more processing units comprises a quantum computer, wherein said quantum computer is configured to evaluate the energy or the force at said coordinate based at least in part on the quantum chemistry calculation corresponding to the training data set of said trained model.
